(19) United States
(12) Patent Application Publication
(10) Pub. No.: US 2025/0032370 A1
(43) Pub. Date: Jan. 30, 2025

(54) CREAMY COSMETIC COMPOSITION

(71) Applicant: SHISEIDO COMPANY, LTD., Tokyo (JP)

(72) Inventor: Satoshi YAMAKI, Yokohama (JP)

(73) Assignee: SHISEIDO COMPANY, LTD., Tokyo (JP)

(21) Appl. No.: 18/715,873

(22) PCT Filed: Dec. 9, 2022
(86) PCT No.: PCT/IB2022/061954

§ 371 Date: Jun. 3, 2024

Related U.S. Application Data
(60) Provisional application No. 63/288,983, filed on Dec. 13, 2021.

(51) Int. Cl. A61K 8/06; A61Q 17/04

(52) U.S. Cl. CPC A61K 8/062; A61Q 17/04; A61K 2800/30; A61K 2800/43; A61K 2800/48; A61K 2800/524

(57) ABSTRACT

An oil-in-water type emulsified cosmetic composition may include (a) a thickener containing at least one natural polymer; and (b) at least one fatty acid: (c) at least one neutralization agent, which partially neutralizes carboxylic acid groups of the at least one fatty acid. The composition does not contain a polyethylene glycol based ingredient.

### CROSS-REFERENCE TO RELATED APPLICATIONS

The present application claims priority under 35 U.S.C. § 371 to International Patent Application No. PCT/IB2022/061954, filed Dec. 9, 2022, which is a Non-Provisional of U.S. Provisional Patent Application No. 63/288,983 filed Dec. 13, 2021. The contents of these applications are hereby incorporated by reference in their entireties.

### FIELD

The present disclosure generally relates to cosmetic compositions and more specifically to cosmetic compositions, which may be creamy and which do not contain certain ingredients, such as a PEG based ingredient, and their methods of making and methods of use.

### SUMMARY

One embodiments is an oil-in-water type emulsified cosmetic composition comprising: (a) at least one fatty acid; and (b) at least one neutralization agent, wherein a mass ratio between the at least one neutralization agent and the at least one fatty acid is from 0.001 to 0.2.

Another embodiment is an oil-in-water type emulsified cosmetic composition comprising: (a) a thickener comprising at least one natural polymer; and (b) at least one fatty acid; (c) at least one neutralization agent, which partially neutralizes carboxylic acid groups of the at least one fatty acid, wherein the composition does not contain a polyethylene glycol ingredient.

### FIGURES

FIGS. 1A-1C show photographs of selected compositions from Examples B-F to demonstrate blurring effect and matteness.

FIGS. 2A-2E show UV-Visible spectra of the composition of selected compsotions.

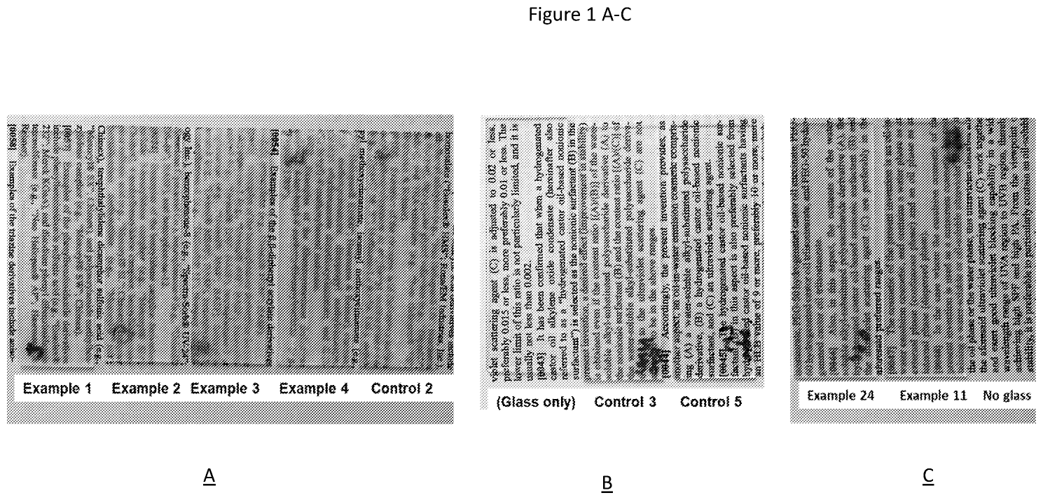

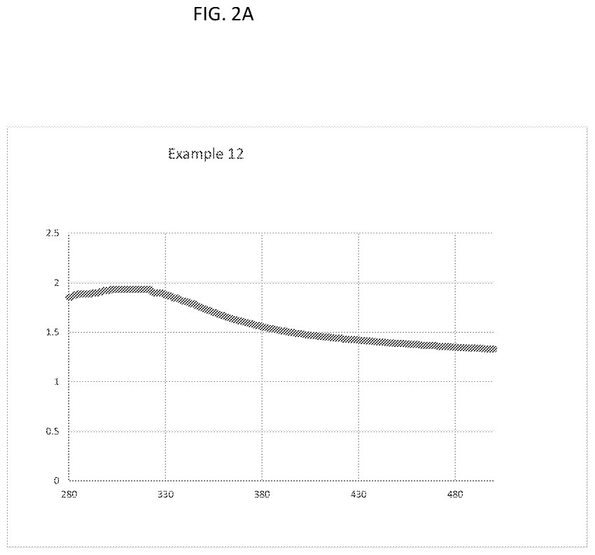

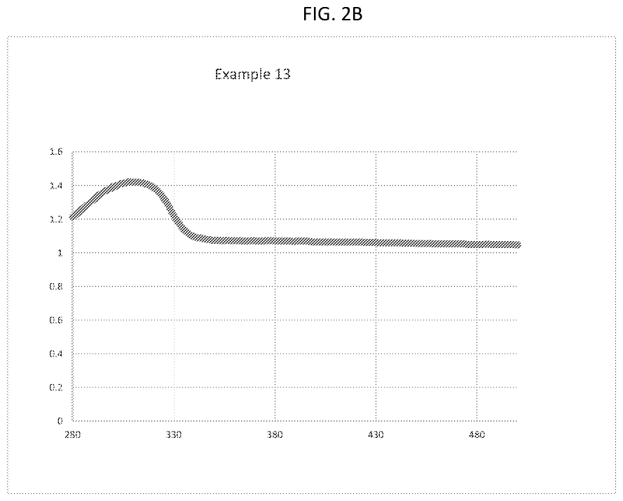

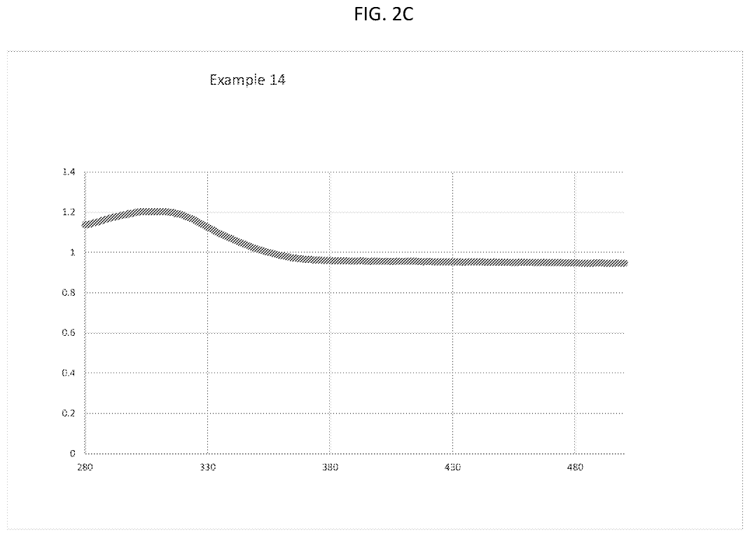

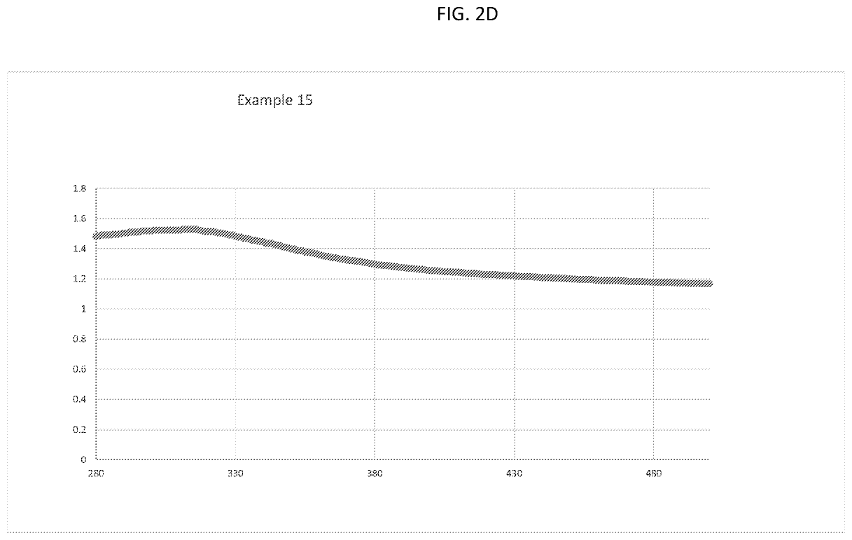

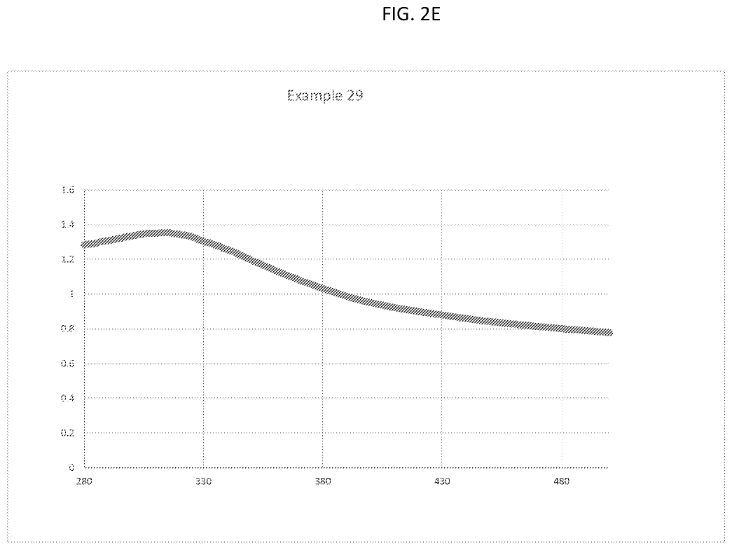

### DETAILED DESCRIPTION

Unless otherwise specified “a” or “an” means one or more.

As used herein, the term “about” placed before a specific numeric value may mean ±20% of the numeric value; ±18% of the numeric value, ±15% of the numeric value; ±12% of the numeric value; ±8% of the numeric value; ±5% of the numeric value; ±3% of the numeric value; ±2% of the numeric value; ±1% of the numeric value or ±0.5% of the numeric value.

All content information for ingredients of compositions expressed as percent (%) refers to percent (%) by mass, relative to the total mass of the composition, unless specified otherwise.

As used herein, the expression “oil-in-water composition” and related expressions refers to an oil-in-water type emulsified composition in which an oil phase is dispersed in an aqueous (water) phase. As used herein, the expression “powder-in-oil-in-water composition” and related expressions refers to an oil-in-water type emulsified composition, in which a powder is dispersed in an oil phase, which is dispersed in an aqueous (water) phase. Powder-in-oil-in-water compositions are disclosed, for example, in WO2017/057563, which is incorporated herein by reference in its entirety.

As used herein, the expression “microplastic beads” refers to particles containing a solid polymer, to which additives or other substances may have been added, and where ≥1% w/w of particles have (i) all dimensions 0.1 μm≤x≤5 mm, or (ii), for fibres, a length of 0.3 μm≤x≤15 mm and length to diameter ratio of >3. The expression “particles containing a solid polymer” refers either (i) a particle of any composition with a continuous solid polymer surface coating of any thickness or (ii) particles of any composition with a solid polymer content of ≥1% w/w.

New standards and regulations regarding safety and non-toxicity as well as customer concerns affected a number of ingredients used in cosmetic compositions. For example, polyethylene glycol (PEG) based ingredients, such as PEG surfactants, silicones, acrylates and/or acrylamide may be undesirable ingredients for cosmetic compositions. However, elimination of these ingredients makes it challenging to make appropriate skincare sensory. For example, it may be difficult to make an oil-in-water type skincare creamy composition with high viscosity and good spreadability without PEG surfactant(s). Without PEG surfactant(s), it may be also difficult to make an oil-in-water type creamy composition or an powder-in-oil-in-water type creamy composition that is stable. Furthermore, without microplastic particles, it may be difficult to produce a cosmetic composition which provides a sufficient blurring effect.

The present disclosure provides a cosmetic composition which may have creamy texture. The creamy texture may mean that when applied to a keratinous surface, such as a skin, the composition smears easily leaving a uniform layer providing a good coverage. The creamy texture is a sensory aspect may relate to a hardness and/or viscosity of the composition.

In many embodiments, such composition may contain no PEG based ingredient, such a PEG surfactant. In many embodiments, the present composition may be free of (meth)acrylates or (meth)acrylamide polymers. In many embodiments, the present composition may contain no PEG surfactant. In many embodiments, the present composition may be free of synthetic polymers. In many embodiments, the present composition may be free of any polyethylene glycol containing ingredients, such as PEG surfactants, any polymeric ingredients, such as (meth)acrylates or (meth)acrylamide; and/or any silicones. In many embodiments, the composition may be free of microplastic particles as defined above. Thus, the present composition may qualify within “clean beauty” cosmetics category.

In some embodiments, the cosmetic composition may have a viscosity of at least 10,000 mPa·s or of at least or more than 15,000 mPa·s or of at least or more than 20,000 mPa·s or any value or subrange within these ranges. In some embodiments, the viscosity may be no greater than 500,000 mPa·s or no greater than 400,000 mPa·s or no greater than 300,000 mPa·s or greater than 200,000 mPa·s or greater than 100,000 mPa·s. The viscosity of the composition may be measured, for example, using a viscometer, such as Brookfield DV-1 viscometer with LV-04 at 12 rpm, for 1 minute.

In some embodiments, the cosmetic composition may have a hardness of at least 4 mN or at least 10 mN or at least 15 mN or at least 20 mN or any value or subrange within these ranges. In some embodiments, the hardness may be no greater than 800 mN or no greater than 700 mN or no greater than 500 mN or no greater than 200 mN.

In some embodiments, the cosmetic composition may have both of the above viscosity and hardness.

In some embodiments, the cosmetic composition may be an oil-in-water type emulsified cosmetic composition comprising: (a) at least one fatty acid; and (b) at least one neutralization agent, wherein a mass ratio between the at least one neutralization agent and the at least one fatty acid is from 0.001 to 0.2 or from 0.005 to 0.18 or 0.01 to 0.15 or from 0.02 to 0.15 or from 0.05 to 0.15 or any value or subrange within these ranges. Such cosmetic composition may have a creamy texture.

Yet in some embodiments, the cosmetic composition may be an oil-in-water type emulsified cosmetic composition comprising: (a) a thickener comprising at least one natural polymer; and (b) at least one fatty acid; (c) at least one neutralization agent, which partially neutralizes carboxylic acid groups of the at least one fatty acid, wherein the composition does not contain a polyethylene glycol ingredient. This means that at least a portion of carboxylic acid groups of the at least one fatty acid is neutralized by the at least one neutralizing agent. Such cosmetic composition may have a creamy texture.

In some embodiments, the cosmetic composition may have sun protection properties. For example, the cosmetic composition may have a sun protection factor SPF at least 2 or at least 5 or at least 10 or at least 20 or at least 30 or at least 40 or at least 50 or at least 60 or at least 70 or at least 80 or at least 90 or at least 100.

The composition may be used in a number of cosmetics products. For example, the present composition may be used in a skin care product, a sun screen product, a primer product; a foundation product, a hair product, a deodorant, a lip product, such as a lipstick or lip balm, a moisturizer, such as a color or tinted moisturizer. In some embodiments, the present composition may be a cream or a cream foundation, which may have sun protection properties.

In some embodiments, the cosmetic composition may be used alone. In other words, the composition may be applied alone, without another composition, to a keratinous surface or substrate, such as skin, e.g. lips, eyelids, face, or hair, of a subject, such as a human.

In some embodiments, the composition, which may be a cosmetic composition, may be used together with another product, for example, a top coat, a primer, or a powder.

### Oil

The oil phase of the composition comprises at least one oil. In some embodiments, the at least one oil may comprise at least one hydrocarbon oil. In some embodiments, the at least one oil may comprise squalane (a triterpene consisting of 2,6,10,15,19,23-Hexamethyltetracosane). In some embodiments, the at least one oil may comprise squalane and at least one alcohol ester, such as an ethylhexyl alcohol ester, which may be for example, ethylhexyl olivate.

In some embodiments, the at least one oil may comprise at least one hydrocarbon.

In some embodiments, the at least one oil solvent may comprise at least one non-polar oil.

A non-polar oil solvent may one or more of non-polar oils disclosed in U.S. Pat. No. 10,154,954, which is incorporated herein by reference it its entirety.

Nonpolar oils are usually hydrocarbons. They lack an electronegative element like oxygen, which results in their typical hydrocarbon feel.

These oils may be of vegetable, mineral or synthetic origin.

The term “non-polar oil” may mean an oil for which the solubility parameter at 25 degrees centigrade, deltaₐ, as defined in U.S. Pat. No. 10,154,954 is equal to 0 (J/cm³)^{1/2}.

The term “hydrocarbon oil” may mean an oil formed essentially from, indeed even composed of, carbon and hydrogen atoms, and optionally oxygen and nitrogen atoms, and not containing any silicon or fluorine atoms. It may contain alcohol, ester, ether, carboxylic acid, amine and/or amide groups.

In some embodiments, the non-polar oil may include one or more non-volatile non-polar hydrocarbon oils.

The non-volatile non-polar hydrocarbon oil may be chosen from linear or branched hydrocarbons of mineral or synthetic origin, such as: liquid paraffin or derivatives thereof, squalane, isoeicosane, naphthalene oil, Isosdodecane, isohexadecane; alkane; polybutylenes such as Indopol H-100 (molar mass or MW=965 g/mol), Indopol H-300 (MW=1340 g/mol) and Indopol H-1500 (MW=2160 g/mol) sold or manufactured by the company Amoco, hydrogenated polyisobutylenes such as Parleam(R) sold by the company Nippon Oil Fats Corporation, Panalane H-300 E sold or manufactured by the company Amoco (MW=1340 g/mol), Viseal 20000 sold or manufactured by the company Synteal (MW-6000 g/mol) or Rewopal PIB 1000 sold or manufactured by the company Witco (MW=1000 g/mol), decene/butene copolymers, polybutene/polyisobutene copolymers, especially Indopol L-14, polydecenes and hydrogenated polydecenes such as: Puresyn 10 (MW-723 g/mol) and Puresyn 150 (MW=9200 g/mol) sold or manufactured by the company Mobil Chemicals, and mixtures thereof.

In some embodiments, the at least one non-polar oil is chosen from hydrogenated polyisobutenes and/or polybutenes.

In some embodiments, the at least one non-polar oil may include squalane (a triterpene consisting of 2,6,10,15,19,23-Hexamethyltetracosane).

In some embodiments, the at least one non-polar oil may include C₉-C₁₄ alkanes, such as isoparaffins, isododecane and isohexadecane. In some embodiments, the at least one non-polar oil may include Vegelight 1214LC, which is C₉₋₁₂ Alkanes (and) Coco-caprylate/caprate.

In some embodiments, the at least one oil solvent may comprise at least volatile oil solvent and at least one non-volatile oil solvent. In some embodiments, the at least one oil solvent may comprise a volatile hydrocarbon oil and a non-volatile hydrocarbon oil.

A volatile oil, such a volatile hydrocarbon oil, refers to an oil, such as hydrocarbon oil, having a boiling point lower than 300° C. at 1 atm. Examples of volatile hydrocarbon oils include, but not limited to, C₉-C₁₄ alkanes, such as isoparaffins, isododecane and isohexadecane. In some embodiments, a volatile hydrocarbon oil may be Vegelight 1214LC, which is C₉₋₁₂ Alkanes (and) Coco-caprylate/caprate.

A non-volatile oil, such as non-volatile hydrocarbon oil, refers to an oil, such as a hydrocarbon oil, having a boiling point of 300° C. or higher at 1 atm, Examples of non-volatile hydrocarbon oils include, but not limited to, light squalane, hydrogenated polydecene and vaseline.

A content of the at least one oil in the composition may vary. In some embodiments, an amount of the at least one oil may be from 0.1 mass % to 50 mass % or from 0.5 mass % to 40 mass % or from 1 mass % to 30 mass % or from 1 mass % to 20 mass % or from 2 mass % to 15 mass % or from 3 mass % to 10 mass % or any value or subrange within these ranges.

### Water

The aqueous phase of the composition comprises water. A content of water in the composition may vary. In some embodiments, an amount of water may be from 1 mass % to 90 mass % or from 5 mass % to 80 mass % or from 10 mass % to 70 mass % or from 30 mass % to 70 mass % or from 35 mass % to 65 mass % or from 40 mass % to 60 mass % or any value or subrange within these ranges. In some embodiments, the composition may comprise one or more hydrophilic glycols, which may reduce water content of the composition.

### Fatty Acid

The least one fatty acid is not particularly limited as long as it is of a type that can be used in cosmetics or the like, and may be selected from among linear or branched fatty acids having saturated or unsaturated hydrocarbon groups.

Particular examples of fatty acids include linear higher fatty acids that are solid at room temperature and that have 8 to 22 carbon atoms such as, for example, lauric acid, myristic acid, palmitic acid, stearic acid, behenic acid and oleic acid. In some embodiments, the at least one fatty acid may include at least one fatty acid selected from the group consisting of stearic acid, palmitic acid and behenic acid.

In some embodiments, at least one fatty acid includes at least one saturated fatty acid or at least one unsaturated fatty acid. In some embodiments, at least one fatty acid may include at least one fatty acid having from about 14 to 22 carbon atoms or from 16 to 22 carbon atoms or from 14 to 20 carbon atoms. In some embodiments, at least one fatty acid may include at least one saturated fatty acid having from about 14 to 22 carbon atoms or from 16 to 22 carbon atoms or from 14 to 20 carbon atoms. In some embodiments, at least one fatty acid may include at least one linear and saturated fatty acid having from about 14 to 22 carbon atoms or from 16 to 22 carbon atoms or from 14 to 20 carbon atoms. In some embodiments, the at least one fatty acid may be selected from lauric acid, tridecylic acid, myristic acid, pentadecylic acid, palmitic acid, margaric acid, stearic acid, nonadecylic acid, arachidic acid, heneicosylic acid, behenic acid, tricosylic acid, lignoceric acid and their combinations. In some embodiments, the at least one fatty acid may comprise stearic acid, behenic acid or their combination.

A content of the at least one fatty acid in the composition may vary. In some embodiments, an amount of the at least one fatty acid may be at least 0.1 mass % or at least 0.5 mass % or at least 1 mass % or at least 1.5 mass % or at least 2 mass % or at least 2.5 mass % or at least 3 mass % of the composition. In some embodiments, an amount of the at least one fatty acid may be from 0.1 mass % to 40 mass % or from 0.5 mass % to 30 mass % or from 1 mass % to 25 mass % or from 1.5 mass % to 20 mass % or from 1 mass % to 8 mass % or from 1.5 mass % to 7 mass % or from 2 mass % to 6 mass % or from 2.5 mass % to 5 mass % or any value or subrange within these ranges.

The partially neutralized fatty acid may act as a surfactant and provide the composition with soapy property.

### Neutralizing Agent

The at least one neutralizing agent may include at least one water soluble base. The at least one water soluble base may include at least one inorganic water soluble base such as ammonium hydroxide, potassium hydroxide or sodium hydroxide. In some embodiments, the at least one neutralizing agent may include at least one of potassium hydroxide and sodium hydroxide.

A content of the at least one neutralizing agent in the composition may vary. In some embodiments, an amount of the at least one neutralizing agent may be from 0.01 mass % to 5 mass % or from 0.02 mass % to 4 mass % or from 0.05 mass % to 3 mass % or from 0.02 mass % to 2 mass % or from 0.05 mass % to 1.25 mass % or from 0.1 mass % to 1 mass % or any value or subrange within these ranges.

The at least one water soluble neutralizing agent may be originally in the water phase of the composition, while the at least at least one fatty acid may be originally in the oil phase of the composition. However, the fatty acid neutralized by the neutralizing agent may be interphase, i.e. between the oil phase and the water phase.

### Natural Polymer

In some embodiments, the composition may include a thickener comprising at least natural polymer, which may be a polymer of natural origin. Natural polymers occur in nature and can be extracted. Non-limiting examples of natural polymers include succinoglycan, sclerotium gum, xanthan gum, a cellulose derivative or a combination thereof.

The cellulose derivative may be an amphiphilic cellulose derivative. In some embodiments, a cellulose derivative may be a hydroxypropyl-methylcellulose alkyl ether, such as a hydroxypropyl-methylcellulose C15-C20 alkyl ether, e.g. hydroxypropyl-methylcellulose stearoxy ether. Such cellulose derivative are disclosed, for example, in US 20120058061, which is incorporated herein by reference in its entirety.

Non-limiting examples of cellulose derivatives include Sangelose 90 L and 60 L (HYDROXYPROPYL METHYLCELLULOSE STEAROXY ETHER) and AQUALON™ CMC 7M2F (cellulose gum).

In some embodiments, the cellulose derivative may be carboxymethyl cellulose also known as cellulose gum.

the cellulose derivative may be hydrophobically Modified Alkylcellulose. The hydrophobically modified alkylcellulose may be one represented by the formula (II).

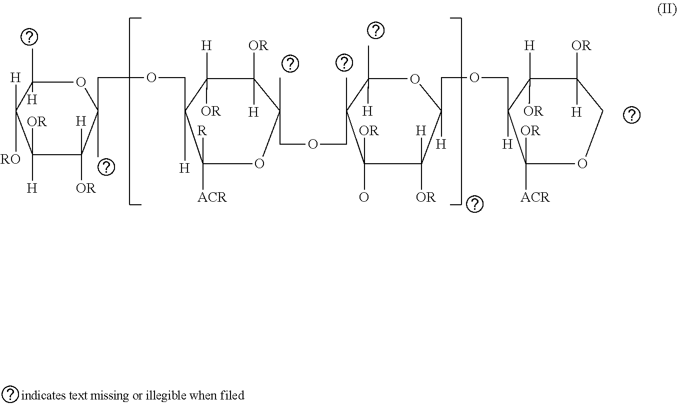

In the above formula (II), R is a bonded (composite) group R₁-R₂, R₁ is the same or different in the molecule, and is a group selected from —[CH₂CH(CH₃)O]ᵣ—, —[CH₂CH₂]ᵣ— and —[CH₂CH(OH)CH₂]ᵣ— (wherein r is an integer of 0 to 4), R₂ is at least one group selected from a hydrocarbon group having 12 to 28 carbon atoms (preferably an alkyl group having 1 to 4 carbon atoms) and a hydrogen atom, and at least one R₂ in a molecule is a hydrocarbon group having 12 to 28 carbon atoms (preferably an alkyl group having 1 to 4 carbon atom). A is a group —(CH₂)ₜ— (wherein t is an integer of 1 to 3) and s is a number of 100 to 10,000.

The hydrophobically modified alkylcellulose of the formula (II) has a structure in which a long chain alkyl group, which is a hydrophobic group, is introduced into a water-soluble cellulose ether derivative through a polyoxyalkylene chain. Examples of water-soluble cellulose ether derivatives, which are the base of the molecule, include methylcellulose, ethyl cellulose, propyl cellulose, butyl cellulose, hydroxyethyl cellulose, Hydroxypropylcellulose and hydroxypropyl methylcellulose. The hydrophobically modified alkylcellulose may be prepared by reacting those with long chain alkyl glycidyl ether (e.g., those represented by the following formula (II′)).

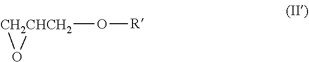

In the formula (II′), R′ is an alkyl group having 10 to 28, preferably 12 to 22 carbon atoms.

Hydroxypropyl methylcellulose or hydroxyethyl cellulose are preferred as the above water-soluble cellulose ether derivative. In particular, it is preferable to select hydroxypropyl methylcellulose. Furthermore, it is preferable that R′ in long chain alkyl glycidyl ether (II′) is a stearyl group (—C₁₈H₃₇) or a cetyl group (—C₁₆H₃₃) (in these cases, —CH₂CH(OH)CH₂OR′ is —CH₂CH(OH)CH₂O—C₁₈H₃₇ or —CH₂CH(OH)CH₂O—C₁₆H₃₃).

One example of the hydrophobically modified alkylcellulose (component B-2) may be stearoxy hydroxypropyl methylcellulose (INCI name: Hydroxypropylmethylcellulose Stearoxy Ether in which the hydrophobic group R′ in the formula (II′) is a stearyl group. A product with the product name “SANGELOSE” commercially available from Daido Chemical Corporation may also be used. (Product names: SANGELOSE 90L, 90M, 90H, 60L, 60M, 60H and the like).

In some embodiments, the composition may include a natural polysaccharide, which may be a natural water soluble polysaccharide, such as succinoglycan.

A content of the at least one natural polymer in the composition may vary in the composition. In some embodiments, an amount of the least one natural polymer, such as succinoglycan and/or a cellulose derivative, which may hydroxypropyl-methylcellulose stearoxy ether, may be from 0.01 mass % to 5 mass % or from 0.03 mass % to 3 mass % or from 0.05 mass % to 2 mass % or from 0.1 mass % to 2 mass or from 0.1 mass % to 1.5 mass % or any value or subrange within these ranges.

In some embodiments, the at least one natural polymer may include a water soluble natural polymer, which may be a part of the water phase of the composition. The water soluble natural polymer may be a water soluble polysaccharide such as succinoglycan.

In some embodiments, the at least one natural polymer may include an amphiphilic natural polymer, which may be a part of both the water phase and the oily phase of composition.

### Powder

In some embodiments, the composition may include one or more powders. For example, the one or more powders may be dispersed in the oily phase. In such case, the composition may be a powder-in-oil-in-water composition. The one or more powders may include one or more surface treated powders, such as one or more hydrophobically surface treated powders. Hydrophobic surface treatment may facilitate dispersion of the powder in the oily phase.

In some embodiments, one or more powders may include hydrophilic powders, which may be in the water phase of the compositions. Non-limiting examples of hydrophilic powders include silica and boron nitride powders.

In some embodiments, one or more powders may include one or more of titanium dioxide particles, silica particle, iron oxide particles and zinc oxide particles. A hydrophobization surface treatment may be applied to one or more powders, such as titanium dioxide particles, silica particles, iron oxide particles and/or zinc oxide particles. Such hydrophobic surface treatment may be (1) a treatment with a metallic soap consisting of a higher fatty acid and a multivalent metal, such as a divalent, e.g. magnesium, or a trivalent metal, e.g. aluminum or (2) a composite treatment with a higher fatty acid and a hydroxide of a multivalent metal, such as a divalent metal, e.g. magnesium or a trivalent metal, e.g. aluminum. The higher fatty acid may be a C8 to C24, such as C12 to C22, linear or branched carboxylic acid, e.g. stearic acid or isostearic acid. For example, in some embodiments, one or more powders, such as titanium dioxide particles, silica particles, iron oxide particles and/or zinc oxide particles, may be surface treated with magnesium stearate and/or magnesium isostearate or with a composite treatment of aluminum hydroxide and stearic and/or isostearic acid.

In some embodiments, one or more powders may provide at least some of sun protection properties, such as ultraviolet protection properties, to the composition. For examples, the one or more powders may comprise one or more types of ultraviolet scattering powders. Examples of ultraviolet scattering powders include titanium dioxide powders, zinc oxide powders, silica powders, and composite powders, such as titanium dioxide coated mica, titanium dioxide coated bismuth oxychloride, titanium dioxide coated talc and titanium dioxide coated glass flake. Ultraviolet scattering powders may have an average particle diameter of about 25 to 100 nm. In some embodiments, one or more ultraviolet scattering powders may include titanium dioxide powder, zinc oxide powder or a combination thereof.

Ultraviolet scattering particles may hydrophobic treated on a surface of a base material, such as zinc oxide or titanium dioxide. Examples of methods of hydrophobic treatment of surface include silicone treatment using methylhydrogen polysiloxane, methyl polysiloxane, trimethylsiloxysilicic acid, silicone resin or the like; fluorine treatment using perfluoroalkyl phosphoric acid ester, perfluoroalcohol or the like; amino acid treatment using N-acylglutamic acid or the like; lecithin treatment; metallic soap treatment; fatty acid treatment; and alkylphosphoric acid ester treatment.

A content of the one or more ultraviolet scattering powders in the composition may vary. In some embodiments, an amount of one or more ultraviolet scattering powders may be from 0.5 mass % to 30 mass % or from 1 mass % to 25 mass % or from 2 mass % to 20 mass % or from 2.5 mass % to 30 mass %, or from 3 mass % to 25 mass % or from 4 mass % to 20 mass % or any value or subrange within these ranges.

### Organic Ultraviolet Absorbers

In some embodiments, the composition may comprise one or more organic ultraviolet absorbers. The one or more organic ultraviolet absorbers may provide at least some of sun protection properties to the composition.

In some embodiments, one or more organic ultraviolet absorbers may include one or more oil-soluble ultraviolet absorbers, which may be dissolved in the oil phase of the composition.

In some embodiments, one or more organic ultraviolet absorbers may include one or more water soluble ultraviolet absorbers, which may be dissolved in the water phase of the composition. A non-limiting example of water soluble ultraviolet absorbers is henylbenzimidazole sulfonic acid.

Examples of oil-soluble ultraviolet absorbers include, but not limited to benzoic acid derivatives, salicylic acid derivatives, cinnamic acid derivatives, dibenzoylmethane derivatives, β-β-diphenyl acrylate derivatives, benzophenone derivatives, benzylidene camphor derivatives, phenylbenzimidazole derivatives, triazine derivatives, phenylbenzotriazole derivatives, anthranil derivatives, imidazoline derivatives, benzal malonate derivatives, and 4,4-diarylbutadiene derivatives.

Examples of benzoic acid derivatives include, but not limited to ethyl p-aminobenzoate (PABA), ethyl dihydroxypropyl PABA, ethylhexyl dimethyl PABA (e.g., “Escalol™. 507; ISP), glyceryl PABA, PEG-25 PABA (e.g., “Uvinul™ P25”; BASF), and diethylamino hydroxybenzoyl hexyl benzoate (e.g., “Uvinul™ A Plus”).

Examples of salicylic acid derivatives include, but not limited to homosalate (“Eusolex™ HMS”; Rona/EM Industries, Inc.), ethylhexyl salicylate (e.g., “Neo Heliopan™ OS”; Haarmann & Reimer), dipropylene glycol salicylate (e.g., “Dipsal™”; Scher), and TEA-salicylate (e.g., “Neo Heliopan™ TS”; Haarmann & Reimer).

Examples of cinnamic acid derivatives include, but not limited to octyl methoxycinnamate or ethylhexyl methoxycinnamate (e.g., “Parsol™ MCX”; Hoffmann-La Roche, Ltd.), isopropyl methoxycinnamate, isoamyl methoxycinnamate (e.g., “Neo Heliopan™E1000”; Haarmann & Reimer), cinoxate, DEA methoxycinnamate, diisopropyl methylcinnamate, glyceryl ethylhexanoate dimethoxycinnamate, and di-(2-ethylhexyl)-4′-methoxybenzal malonate.

Examples of dibenzoylmethane derivatives include, but not limited to 4-tert-butyl-4′-methoxydibenzoylmethane (e.g., “Parsol™ 1789”).

Examples of β-β-diphenyl acrylate derivatives include octocrylene (e.g., Uvinul™ N539”; BASF).

Examples of benzophenone derivatives include benzophenone-1 (e.g., “Uvinul™ 400”; BASF), benzophenone-2 (e.g., “Uvinul™ D50”; BASF), benzophenone-3 or oxybenzone (e.g., “Uvinul™ M40”; BASF), benzophenone-4 (e.g., “Uvinul™ MS40”; BASF), benzophenone-5, benzophenone-6 (e.g., “Helisorb™ 11”; Norquay Technology Inc.), benzophenone-8 (e.g., “Spectra-Sorb™ UV-24”; American Cyanamid Co.), benzophenone-9 (e.g., “Uvinul™DS-49”; BASF), and benzophenone-12.

Examples of the benzylidene camphor derivatives include 3-benzylidene camphor (e.g., “Mexoryl™ SD”; Chimex), 4-methylbenzylidene camphor, benzylidene camphor sulfonic acid (e.g., “Mexoryl™ SL”; Chimex), camphor benzalkonium methosulfate (e.g., “Mexoryl™ SO”; Chimex), terephthalylidene dicamphor sulfonic acid (e.g., “Mexoryl™ SX”; Chimex), and polyacrylamide methylbenzylidene camphor (e.g., “Mexoryl™ SW”; Chimex).

Examples of phenylbenzimidazole derivatives include phenylbenzimidazole sulfonic acid (e.g., “Eusolex™ 232”; Merck KGaA), and disodium phenyl dibenzimidazole tetrasulfonate (e.g., “Neo Heliopan™ AP”; Haarmann & Reimer).

Examples of triazine derivatives include anisotriazine (e.g., “Tinosorb™ S”; Ciba Specialty Chemicals Inc.), ethylhexyl triazone (e.g., “Uvinul™ T-150”; BASF), diethylhexyl butamido triazone (e.g., “Uvasorb™ HEB”; 3V SIGMA S.p.A.), and 2,4,6-tris(diisobutyl-4′-aminobenzalmalonate)-s-triazine.

Examples of phenylbenzotriazole derivatives include drometrizole trisiloxane (e.g., “Silatrizole™ Rhodia Chimie), and methylene bis-benzotriazolyl tetramethylbutylphenol (e.g., “Tinosorb™ M”; Ciba Specialty Chemicals Inc.).

Examples of anthanil derivatives include menthyl anthranilate (e.g., “Neo Heliopan™ MA”; Haarmann & Reimer).

Examples of imidazoline derivatives include ethyihexyl dimethoxybenzylidene dioxoimidazoline propionate.

Examples of the benzal malonate derivatives include polyorganosiloxane having a benzal malonate functional group (e.g., Polysilicone-15; “Parsol™ SLX”; DSM Nutrition Japan K.K.).

Examples of the 4,4-diarylbutadiene derivatives include 1,1-dicarboxy (2,2′-dimethylpropyl)-4,4-diphenylbutadiene.

A content of the one or more organic ultraviolet absorbers, such as one or more oil soluble ultraviolet absorbers, may vary. In some embodiments, the composition may contain no such absorbers at all. In such case, sun protection properties, such as ultraviolet protection properties, of the composition may be due to ultraviolet scattering powder(s). Yet in some embodiments, the composition may contain the one or more organic ultraviolet absorbers, such as one or more oil soluble ultraviolet absorbers, in an amount from 0.5 mass % to 30 mass % or from 1 mass % to 25 mass % or from 3 mass % to 20 mass % or 5 mass % to 18 mass % or any value or subrange within those ranges.

### Pigments

In some embodiments, the composition may also include one or more pigments.

In some embodiments, the one or more pigments may comprise one or more water dispersible pigments. Water dispersible pigments may be dispersed in the aqueous phase of the composition. Water dispersible pigments may have be hydrophilically surface treated pigments, i.e. pigments have their outer surface hydrophobically treated. In some embodiments, the one or more water dispersible pigments may comprise one or more mineral pigments, which may have their surface hydrophilically treated for example, with an outer layer of silica. Thus, in some embodiments, the one or more water dispersible pigments may comprise one or more silica covered pigments, such as silica covered iron oxide and silica covered titanium dioxide. Silica-covered iron oxide pigments, such as PRIMROSE-Y-77492; PRIMROSE-R-77491; PRIMROSE-B-77499, and silica covered titanium dioxide pigments, such as PRIMROSE-TRI-77891, are commercially available from Miyoshi, America, Inc.

In some embodiments, the one or more pigments may comprise one or more hydrophobically surface treated pigments, i.e. pigments have their outer surface hydrophobically treated. Hydrophobically surface treated pigments may be dispersed in the oily phase of the composition. In some embodiments, the one or more hydrophobically surface treated pigments may comprise one or more mineral pigments, such as iron oxide pigments, may be hydrophobically surface treated with, for example, lauroyl lysine. Lauroyl lysine treated iron oxide pigments are commercially available, for example, as Unipure Yellow LC 182 LL, Unipure Red LC 381 LL and Unipure Black LC 989 LL.

Additional examples of pigments include ASL treated pigments from Daito Kasei, such as titanium oxide and/or iron oxide with sodium lauroyl glutamate, lysine and magnesium chloride, NHS treated pigments from Miyoshi, such titanium oxide and/or iron oxide with isostearyl sebacare, disodium stearoyl glutamate and aluminum hydroxide.

In some embodiments, one or more pigments may be in a form of a powder, which may be a hydrophobically surface treated powder, a hydrophilically surface treated powder or a combination thereof. In some embodiments, a powder used as a pigment may be a pigment grade powder. The term “pigment grade” may mean an average particle size in the powder of about 0.2 to 0.4 μm. In some embodiments, one or more pigments may be selected from pigment-grade titanium dioxide, pigment-grade zinc oxide, fine particle zinc oxide, talc, mica, sericite, kaolin, titanated mica, black iron oxide, yellow iron oxide, red iron oxide, ultramarine, Prussian blue, chromium oxide, chromium hydroxide, silica and cerium oxide. In some embodiments, one or more pigments may include one or more mineral pigments, such as one or more iron oxide pigments, such as black iron oxide, yellow iron oxide and red iron oxide.

A content of one or more pigments in the composition may vary. For example, in some embodiments, an amount of one or more pigments in the composition may be from 0.05 mass % to 25 mass % or from 0.1 mass % to 20 mass % or from 0.2 mass % to 18 mass % or from 0.3 mass % to 15 mass % or from 0.1 mass % to 15 mass % or from be from 0.2 mass % to 10 mass % or from 0.3 mass % to 8 mass % or from 0.5 mass % to 5 mass % or 0.3 mass % to 3 mass % or any value or subrange within these ranges.

### Amphiphilic Agent

In some embodiments, the composition may include at least one non-PEG water soluble amphiphilic agent. In some embodiments, the at least one non-PEG water soluble amphiphilic agent may comprise at least one fatty alcohol.

In some embodiments, the at least one fatty alcohol may be selected from linear or branched, saturated or unsaturated C14-C30 or C16-C26 or C18-C24 fatty alcohols.

In some embodiments, the at least one fatty alcohol may be selected from linear and saturated C14-C30 fatty alcohols or linear and saturated C16-C24 fatty alcohols or linear and saturated C18-C24 fatty alcohols. In one exemplary embodiment, the at least one fatty alcohol may comprise behenyl alcohol.

An content of the at least one non-PEG water soluble amphiphilic agent, such as a fatty alcohol, e.g. behenyl alcohol, in the composition may vary. In some embodiments, an amount of the at least one non-PEG water soluble amphiphilic agent, such as a fatty alcohol, e.g. behenyl alcohol, in the composition may be from 0.01 mass % to less than 1 mass % or from 0.02 mass % to 3 mass % or from 0.03 mass % to 2.5 mass % or from 0.05 mass % to 2 mass % or from 0.01 mass % to less 1 mass % or from 0.02 mass % to 0.95 mass % or from 0.03 mass % to 0.9 mass % or from 0.1 mass % to 0.85 mass % or any value or subrange within these ranges. Having less than 1 mass % of the at least one non-PEG water soluble amphiphilic agent, such as a fatty alcohol may provide the composition with less soapy white cast. Without low-surface-tension silicone oils, the cream with a soap composition can easily cause soapy white cast when applied to a keratinous surface, such as a skin. The soapy white cast is generally not preferable because consumers usually prefer the transparent appearance with the sense of quick absorption into skin. In the present composition, the white cast may be decreased by limiting the amount of the at least one non-PEG water soluble amphiphilic agent, such as fatty alcohol, in the composition.

The use of the at least one fatty alcohol may provide the composition with cream sensory and/or water repellent properties.

The at least one fatty alcohol may be a part of the oil phase of the oil-in-water composition. The at least one fatty alcohol may stabilize the oil-in-water composition.

### Preservative Agent

In some embodiments, the composition may include at least one preservative agent. In some embodiments, the at least one preservative agent may include at least one amphiphilic liquid preservative agent, such as methylheptylglycerin and/or ethylheptylglycerin. Due to the presence of the at least one amphiphilic liquid preservative agent, such as methylheptylglycerin and/or ethylheptylglycerin, the composition may have a decreased white cast. The decrease of white cast may be achieved without using silicone(s) in the composition.

A content of the at least one amphiphilic liquid preservative agent, such as methylheptylglycerin and/or ethylheptylglycerin, in the composition may vary. In some embodiments, an amount of the at least one amphiphilic liquid preservative agent, such as methylheptylglycerin and/or ethylheptylglycerin, may be 0.01 mass % to 5 mass % or from 0.05 mass % to 4 mass % or from 0.1 mass % to 3 mass % or from 0.3 mass % to 2.5 mass % or from 0.5 mass % to 2 mass % or any value or subrange within these ranges.

In some embodiments, the at least one preservative agent may include phenoxyethanol. A content of phenoxyethanol in the composition may vary. In some embodiments, an amount of phenoxyethanol may be from 0.01 mass % to 1.5 mass % or from 0.05 mass % to 1.5 mass % or from 0.05 mass % to 1.2 mass % or from 0.1 mass % to 1.2% or from 0.1 mass % to 1.0 mass % or any value or subrange within these ranges.

### Additional Ingredients

In some embodiments, the composition may also comprise one or more additional ingredient, which may a humectant, an emollient and/or a moisturizer. In some embodiments, the one or more additional ingredient may include glycerin. Glycerin may be in the aqueous phase of the composition. Glycerin may serve as humectant to moisturize skin. A content of glycerin in the composition may vary. In some embodiments, an amount of glycerin may be from 0.1 mass % to 30 mass % or from 0.5 mass % to 20 mass % or from 1 mass % to 15 mass % or from 1 mass % to 10 mass % or from 2 mass % to 8 mass % or 3 mass % to 8 mass % or any value or subrange within these ranges.

In some embodiments, the composition may include 0.1 mass % to 40 mass % or 1 mass % to 8 mass % of at least one of stearic acid and behenic acid; 0.01 mass % to 5 mass % or 0.05 mass % to 1.25 mass % of at least one of potassium hydroxide or sodium hydroxide; 0.01 mass % to 5 mass % or 0.1 mass % to 1 mass of a fatty alcohol; 0.01 mass % to 5 mass % or 0.1 mass % to 2 mass % of succinoglycan; 0.01 mass % to 5 mass % or 0.3 mass % to 3 mass % of methylheptylglycerin, ethylheptylglycerin or a combination thereof; 0.01 mass % to 1.5 mass % or 0.05 mass % to 1.5 mass % of phenoxyethanol; and at least one of (i) 0.5 mass % to 30 mass % or 4 mass % to 20 mass % of titanium oxide particles, zinc oxide particles or a combination thereof; (ii) 0.5 mass % to 30 mass % or 1 mass % to 25 mass % of one or more organic UV absorbers and (iii) 0.05 mass % to 25 mass % or 0.3 mass % to 3 mass % of pigments. The composition may also include 40 mass % to 60 mass % of water.

The composition may be prepared by preparing a water phase of the composition and an oil phase of the composition separately; then adding the oil phase to the water phase and emulsifying the addition product, using for example, an homogenizer. Additional ingredients such as pigments and/or powders, such as UV scattering powders may be added before or after the adding the oil phase to the water phase and emulsifying the addition product. For example, in some embodiments, hydrophobic additional ingredients, such as hydrophobic pigments and/or hydrophobic powders may be added to the oil phase before it is added to the water phase. Similarly, in some embodiments, hydrophilic and/or water dispersible additional ingredients, such as hydrophilic and/or water dispersible pigments and/or hydrophilic and/or water dispersible powders, may be added to the water phase before oil phase is added to the water phase. Yet in some embodiments, additional ingredients such as pigments and/or powders, such as UV scattering powders, to the emulsified product after emulsifying the addition product.

Embodiments described herein are further illustrated by, though in no way limited to, the following working examples.

### Example A

TABLE 1
Ingredients of Exemplary compositions

| Material name En | INCI Name | Ex. X | Ex. Y | Ex. Z |
|---|---|---|---|---|
| DEIONIZED WATER | WATER | To 100 | To 100 | To 100 |
| GLYCERINE NAT 99.7% ACIDCHEM | GLYCERIN | 5 | 5 | 5 |
| CAUSTIC POTASH DRY | POTASSIUM HYDROXIDE & WATER | 0.5 | 0.5 | 0.5 |
| STEARIC ACID POFAC | STEARIC ACID | 4 | 3 | 3 |
| NAA-222S BEADS (JSQI) | BEHENIC ACID | 1 | 0.8 | 0.7 |
| LANETTE ® 22 | BEHENYL ALCOHOL | 0.5 | 0.4 | 0.3 |
| NEO HELIOPAN OS OCTISLTE | ETHYLHEXYL SALICYLATE | 5 | 5 | 5 |
| PARSOL ® 340 | OCTOCRYLENE | 5 | 5 | 5 |
| NEO HELIOPAN HMS HOMSLTE | HOMOSALATE | 1.5 | — | — |
| BOTANESSENTIAL OLIVE S-EHO | ETHYLHEXYL OLIVATE, SQUALANE | 5 | 5 | 5 |
| TITANIUM DIOXIDE MT-100 TV | Titanium Dioxide & Aluminum | 5 | 5 | 5 |
| | Hydroxide & Stearic Acid | | | |
| PRIMROSE-TRI-77891 | TITANIUM DIOXIDE & SILICA | 7 | 7 | 7 |
| PRIMROSE-Y-77492 | IRON OXIDES & SILICA | 1 | 1 | 1 |
| PRIMROSE-R-77491 | IRON OXIDES & SILICA | 0.3 | 0.3 | 0.3 |
| PRIMROSE-B-77499 | IRON OXIDES & SILICA | 0.1 | 0.1 | 0.1 |
| ZEMEA | PROPANEDIOL | 10 | 10 | 10 |
| PHENOXETOL NF 272556 | PHENOXYETHANOL | 0.8 | 0.8 | 0.8 |
| Lexgard Natural MHG MB | Methylheptylglycerin | — | — | — |
| RHEOZAN | SUCCINOGLYCAN | 0.5 | 0.5 | 0.5 |

| Hardness (8 ϕ), mN | 65 | 22 | — |
|---|---|---|---|
| Viscosity (#4,12 prm) mPa s | — | — | 23550 |
| 1M Stability | 0 No | 0 No | |
| | issue | issue | |

The compositions of Table 1 were prepared using the following manufacturing Process:
1. Mix the water phase and water thickener and heated up to 80C (Except yellow and blue columns)
2. Add oil phase (Yellow and blue columns in the chart.)
3. Emulsify by a Disper with 3000 rpm
4. Cooling down to 40C

The composition were evaluated for the following parameters.

### 1. Hardness

Measured the hardness by SUN RHEOMETER COMPAC-100II (Sun SCIENTIFIC CO., LTD.) and its 8Φ attachment

### 2. Viscosity

Measured viscosity by BLOOKFIELD DV-1 Viscometer with LV-04, 12 rpm, If the viscosity is less than 50,000

### 3. Spreadability

+: More than 70% of 10 subjects felt spreading well

+/−: More than 40% of 10 subjects felt spreading well

−: Less than 40% of 10 subjects felt the spreading well

### 4. Soapy White Cast

+: More than 70% of 10 subjects did not mind soapy white cast

+/−: More than 40% of 10 subjects did not mind soapy white cast

−: Less than 40% of 10 subjects did not mind soapy white cast

Behenyl alcohol provides the composition with cream sensory and water repellency.

Due to the presence of ultraviolet scaterring titanium dioxide & aluminum hydroxide & stearic Acid, the compositions of the Examples are stable powder-in-oil-in-water compositions with UV filters.

The presence of PRIMROSE-TRI-77891; PRIMROSE-Y-77492; PRIMROSE-R-77491 and PRIMROSE-B-77499 indicates that the compositions of the Examples are compatible with water-dispersible pigments.

Addition of methylheptylglycerin to the composition of example 3 resulted in a decrease of white cast in that composition, which was achieved without using silicone in the composition.

The compositions of the Examples have a good Spreadability.

The compositions of the Examples can be applied as a creamy SPF tinted moisturizer with skincare sensory.

### Example B

A number of compositions were produced to test the effect of a ratio of fatty acids/neutralization on stability of oil-in-water (O/W) or powder-in-oil-in-water emulsions with high viscosity/hardness and good blurring function without a PEG surfactant. The tested compositions are summarized in Table 2.

The compositions were produced by the following manufacturing process: 1. mix a water phase and a water thickener and heat up to 80C; 2. add an oil phase to the water phase; 3. emulsify the addition product by a Disper with 3000 rpm; 4. cool down to 40C.

The following properties were evaluated in the compositions:

### Evaluation

1. Ratio of neutralization agent/fatty acids=(% of Neutralization)/(% of total fatty acids)
2. Appearance: Visually assessed the condition of samples
3. Hardness: Measured the hardness by SUN RHEOMETER COMPAC-100II (Sun SCIENTIFIC CO., LTD.) and its 8Φ attachement
4. Blurring effect: Cast samples with 70-micrometer thickness on the glass plates and assessed the blurring effects on the paper with character strings
   +: Highly blur the words
   +/−: Blur the words
   −: Did not or tiny blur the words

TABLE 2

| | | | Exam- | Exam- | Exam- | Exam- | |
|---|---|---|---|---|---|---|---|
| Material name En | INCI Name | Control 1 | ple 1 | ple 2 | ple 3 | ple 4 | Control 2 |
| DEIONIZED WATER | WATER | To 100 | To 100 | To 100 | To 100 | To 100 | To 100 |
| DIPROPYLENE GLYCOL | DIPROPYLENE GLYCOL | 10 | 10 | 10 | 10 | 10 | 10 |
| FRAGRANFCE GRADE | | | | | | | |
| CAUSTIC POTASH DRY | POTASSIUM HYDROXIDE & WATER | | 0.1 | 1 | 1.8 | 2.3 | 3 |
| STEARIC ACID POFAC | STEARIC ACID | 15 | 15 | 15 | 15 | 15 | 15 |
| LANETTE ® 22 | BEHENYL ALCOHOL | 2 | 2 | 2 | 2 | 2 | 2 |
| PRIPURE 3759-LQ-(GD) | SQUALANE | 5 | 5 | 5 | 5 | 5 | 5 |
| Property | Ratio of Neutralization agent/fatty acids | 0 | 0.01 | 0.07 | 0.12 | 0.15 | 0.2 |
| | Appearance | Phase | Uniform | Uniform | Uniform | Uniform | Uneven |
| | | separation | cream | cream | cream | cream | cream |
| | Hardness (mN) | 0 | 27 | 39 | 128 | 49 | 4 |
| | Blurring effect | N/A | +/− | + | + | + | + |

Results of Table 2: Achieved uniform creams with good blurring effects by the special ratio of Neutralization agent/Fatty acid without PEG, Silicone, and methyl metacrylate crosspolymer (MPB).

Table 3 presents control composition 3, which unlike the compositions in Table 2, included PEG, Silicone, and MPB, and control composition 4, which does not include any of PEG, or MPB. Control compositions 3 and 4 were manufactured using the manufacturing process outlined above.

TABLE 3

| Material name En | INCI Name | Control 3 | Control 4 |
|---|---|---|---|
| DEIONIZED WATER | AQUA | To 100 | To 100 |
| DM-FLUID-A-6T (KF-96A-6T) | DIMETHICONE | 5 | 5 |
| X-21-5595 | TRIMETHYLSILOXYSILICATE, ISODODECANE | 3 | 3 |
| ABIL WAX 9801 | CETYL DIMETHICONE | 3 | 3 |
| MYRITOL ® 318 | CAPRYLIC/CAPRIC TRIGLYCERIDE | 5 | 5 |
| BELSIL ® DM 1 PLUS | DIMETHICONE | 10 | 10 |
| FLORAESTERS IPJ | JOJOBA ALCOHOL, ISOPROPYL JOJOBATE, | 3 | 3 |
| | JOJOBA ESTERS, TOCOPHEROL | | |
| KF-6051 | PEG-10 DIMETHICONE, TOCOPHEROL | 3 | |
| BENTONE 38VCG | DISTEARDIMONIUM HECTORITE | 2 | 2 |
| GANZPEARL GMX-0810 | METHYL METHACRYLATE CROSSPOLYMER | 4 | |
| EDTA 3•NA2•H20 | TRISODIUM EDTA | 0.2 | 0.2 |
| POLYETHYLENE GLYCOL 20000 | PEG-400 | 3 | |
| GLYCERINE NAT 99.7% ACIDCHEM | GLYCERIN | 3 | 3 |
| DIPROPYLENE GLYCOL FRAGRANCE GRADE | DIPROPYLENE GLYCOL | 5 | 5 |
| 1,3 BUTYLENE GLYCOL | BUTYLENE GLYCOL | 5 | 5 |
| PHENOXETOL NF 272556 | PHENOXYETHANOL | 0.5 | 0.5 |
| Property | Appearance | Uniform | Phase |
| | | cream | separation |
| | Hardness (mN) | 15 | 0 |
| | Blurring effect | +/− | − |

Results of Table 3: Eliminating PEG and MPB ingredients from common formula could not achive uniform cream with hardness and good blurring effect.

Table 4 presents control composition 5, which includes PEG, and control composition 6, which does not include PEG. Control compositions 5 and 6 were manufactured using the manufacturing process outlined above.

TABLE 4

| Material name En | INCI Name | Control 5 | Control 6 |
|---|---|---|---|
| DEIONIZED WATER | AQUA | To 100 | To 101 |
| EDTA-2NA•2H20 | DISODIUM EDTA | 0.1 | 0.1 |
| ZEMEA | PROPANEDIOL | 3.0 | 3.0 |
| PHENOXETOL NF 27255613162 | PHENOXYETHANOL | 1.0 | 1.0 |
| 1,3 BUTYLENE GLYCOL | BUTYLENE GLYCOL | 4.0 | 4.0 |
| KELTROL (RM-09121) | XANTHAN GUM | 0.2 | 0.2 |
| 1,3 BUTYLENE GLYCOL | BUTYLENE GLYCOL | 3.0 | 3.0 |
| MACBIOBRIDE E-1060 | PEG/PPG-14/7 DIMETHYL ETHER | 2.0 | |
| SILICABEADS SB-300 | SILICA | 1.0 | 1.0 |
| AMIHOPE LL | LAUROYL LYSINE | 0.5 | 0.5 |
| SEPINOV EMT10 | HYDROXYETHYL ACRYLATE/SODIUM | 2.6 | |
| | ACRYLOYLDIMETHYL TAURATE | | |
| | COPOLYMER, POLYSORBATE 60, | | |
| | SORBITAN ISOSTEARATE, AQUA | | |
| EASYNOV | OCTYLDODECANOL, OCTYLDODECYL | 2.0 | |
| | XYLOSIDE, PEG-30 | | |
| | DIPOLYHYDROXYSTEARATE, AQUA, | | |
| | XYLOSE | | |
| JOJOBA OIL | SIMMONDSIA CHINENSIS SEED OIL | 2.0 | 2.0 |
| RAIN FOREST 03410 | THEOBROMA GRANDIFLORUM SEED | 5.0 | 5.0 |
| (REFINED CUPUAçU BUTTER) | BUTTER, TOCOPHEROL | | |
| Property | Appearance | Uniform | Phase |
| | | cream | separation |
| | Hardness (mN) | 14 | 0 |
| | Blurring effect | — | — |

Results of Table 4: Eliminating PEG ingredients from common formula could not achive uniform cream with hardness.

### Example C

A number of compositions were prepared to test the effect of natural polymers on spreadability of compositions. The tested compositions are summarized in Tables 5 and 6.

The compositions were produced by the following manufacturing process: 1. Mix a water phase and a water thickener and heat up to 80C; 2. Add an oil phase to the water phase; 3. Emulsify the addition product by a Disper with 3000 rpm; 4. Cool down to 40C.

The compositions were evaluated for a number of properties.

### Evaluation

1. Ratio of neutralization agent/fatty acid=(% of Neutralization)/(% of total fatty acids)
2. Appearance: Visually assessed the condition of samples
3. Hardness: Measured the hardness by SUN RHEOMETER COMPAC-100II (Sun SCIENTIFIC CO., LTD.) and its 8Φ attachement
4. Blurring effect: Cast samples with 70-micrometer thickness on the glass plates and assessed the blurring effects on the paper with character strings

+: Highly blur the words

+/−: Blur the words

−: Did not or tiny blur the words

### 5. Spreadability

+: More than 70% of 10 subjects felt spreading well

+/−: More than 40% of 10 subjects felt spreading well

−: Less than 40% of 10 subjects felt the spreading well

TABLE 5

| Material name En | INCI Name | Control 7 | Example 5 | Control 8 | Example 6 |
|---|---|---|---|---|---|
| DEIONIZED WATER | WATER | To 100 | To 100 | To 100 | To 100 |
| DIPROPYLENE GLYCOL FRAGRANFCE GRADE | DIPROPYLENE GLYCOL | 10 | 10 | 10 | 10 |
| SANGELOSE 60L | HYDROXYPROPYL METHYLCELLULOSE | 0.3 | 0.3 | 0.3 | |
| | STEAROXY ETHER | | | | |
| CAUSTIC POTASH DRY | POTASSIUM HYDROXIDE & WATER | | 2 | 3 | 2 |
| STEARIC ACID POFAC | STEARIC ACID | 15 | 15 | 15 | 15 |
| LANETTE ® 22 | BEHENYL ALCOHOL | 2 | 2 | 2 | 2 |
| PRIPURE 3759-LQ-(GD) | SQUALANE | 5 | 5 | 5 | 5 |
| Property | Ratio of Neutralization agent/fatty acids | 0 | 0.13 | 0.20 | 0.13 |
| | Appearance | Phase | Uniform | Uneven | Uniform |
| | | separation | cream | cream | cream |
| | Hardness (mN) | 6 | 20 | 0 | 49 |
| | Blurring effect | N/A | +/− | + | + |
| | Spreadability | N/A | + | + | +/− |

Results of Table 5: Adding natural polymer maintain the blurring effect and improve the spreadability.

TABLE 6

| | | Exam- | Exam- | Exam- | Exam- | Exam- |
|---|---|---|---|---|---|---|
| Material name En | INCI Name | ple 7 | ple 8 | ple 9 | ple 10 | ple 11 |
| DEIONIZED WATER | WATER | To 100 | To 100 | To 100 | To 100 | To 100 |
| GLYCERINE NAT 99.7% ACIDCHEM | GLYCERIN | 5 | 5 | 5 | 5 | 5 |
| ZEMEA | PROPANEDIOL | 10 | 10 | 10 | 10 | 10 |
| PHENOXETOL NF 272556 | PHENOXYETHANOL | 0.5 | 0.5 | 0.5 | 0.5 | 0.5 |
| SANGELOSE 90L | HYDROXYPROPYL METHYLCELLULOSE | | 0.5 | | | |
| | STEAROXY ETHER | | | | | |
| SANGELOSE 60L | HYDROXYPROPYL METHYLCELLULOSE | | | 0.5 | | |
| | STEAROXY ETHER | | | | | |
| AQUALON ™ CMC 7M2F | CELLULOSE GUM | | | | 1 | |
| RHEOZAN | SUCCINOGLYCAN | | | | | 1 |
| CAUSTIC POTASH DRY | POTASSIUM HYDROXIDE & WATER | 0.5 | 0.5 | 0.5 | 0.5 | 0.5 |
| STEARIC ACID POFAC | STEARIC ACID | 8 | 8 | 8 | 8 | 8 |
| NAA-222S BEADS (JSQI) | BEHENIC ACID | 2 | 2 | 2 | 2 | 2 |
| LANETTE ® 22 | BEHENYL ALCOHOL | 1 | 1 | 1 | 1 | 1 |
| ESERAN 200 | DIISOPROPYL SEBACATE | 5 | 5 | 5 | 5 | 5 |
| NEO HELIOPAN OS OCTISLTE | ETHYLHEXYL SALICYLATE | 5 | 5 | 5 | 5 | 5 |
| DEIONIZED WATER (0.5% Extra water) | WATER | 0.5 | 0.5 | 0.5 | 0.5 | 0.5 |
| Property | Ratio of Neutralization agent/fatty acids | 0.17 | 0.17 | 0.17 | 0.17 | 0.17 |
| | Appearance | Uniform | Uniform | Uniform | Uniform | Uniform |
| | | cream | cream | cream | cream | cream |
| | Hardness (mN) | 49 | 127 | 157 | 25 | 138 |
| | Blurring effect | + | + | + | + | + |
| | Spreadability | +/− | + | + | + | + |

Results of Table 6: Adding natural polymer maintain the blurring effect and improve the spreadability even though the hardness got higher.

### Example D

A number of compositions were tested to incorporate inorganic UV filters (scattering UV powders/particles) and/or pigments. Such compositions may be useful for sun protection and/or color design. The tested compositions are presented in Tables 7-9.

The compositions were produced by the following manufacturing process: 1. Mix a water phase and a water thickener and heat up to 80C; 2. Disperse inorganic UV filters in an oil phase; 3. Add the oil phase to the water phase. 4. Emulsify the addition product by a Disper with 3000 rpm; 5. Add pigments and mix; 6. Cooling down to 40C.

The compositions were evaluated for a number of properties.

### Evaluation

1. Ratio of neutralization agent/fatty acids=(% of Neutralization)/(% of total fatty acids)
2. Appearance: Visually assessed the condition of samples
3. Hardness: Measured the hardness by SUN RHEOMETER COMPAC-100II (Sun SCIENTIFIC CO., LTD.) and its 8Φ attachement
4. Viscosity: Measured viscosity by BLOOKFIELD DV-1 Viscometer with LV-04, 12 rpm, If the viscosity is less than 50,000
5. In vitro UV protective effect: Applied 2 mg/cm2 on PMMA plate (SPF MASTER PA-01), and measure the absorbance from 500 to 280 nm by Hitachi U-3900H Spectrophotometer

+: Absorbance is more than 1.5 at 320 nm

+/−: Absorbance is 1.0 to 1.5 at 320 nm

−: Absorbance is less than 1.0 at 320 nm

6. Matteness: Cast Samples with 70-Micrometer Thickness on the Glass Plates and Assessed the Blurring Effects on the Paper with Character Strings

+: More than 70% of 10 subjects felt matte finish

+/−: More than 40% of 10 subjects felt matte finish

−: Less than 40% of 10 subjects felt matte finish

### 7. Spreadability

+: More than 70% of 10 subjects felt spreading well

+/−: More than 40% of 10 subjects felt spreading well

−: Less than 40% of 10 subjects felt the spreading well

### 8. Soapy White Cast

+: More than 70% of 10 subjects did not mind soapy white cast

+/−: More than 40% of 10 subjects did not mind soapy white cast

−: Less than 40% of 10 subjects did not mind soapy white cast

TABLE 7

| Material name En | INCI Name | Example 12 | Example 13 |
|---|---|---|---|
| DEIONIZED WATER | WATER | To 100 | To 100 |
| GLYCERINE NAT 99.7% ACIDCHEM | GLYCERIN | 5 | 5 |
| CAUSTIC POTASH DRY | POTASSIUM HYDROXIDE & WATER | 0.5 | 0.5 |
| STEARIC ACID POFAC | STEARIC ACID | 4 | 3 |
| NAA-222S BEADS (JSQI) | BEHENIC ACID | 1 | 0.75 |
| LANETTE ® 22 | BEHENYL ALCOHOL | 1 | 0.35 |
| NEO HELIOPAN OS OCTISLTE | ETHYLHEXYL SALICYLATE | 5 | 5 |
| PARSOL ® 340 | OCTOCRYLENE | 5 | 5 |
| NEO HELIOPAN HMS HOMSLTE | HOMOSALATE | 1 | 5 |
| BOTANESSENTIAL OLIVE S-EHO | ETHYLHEXYL OLIVATE, SQUALANE | 5 | |
| TITANIUM DIOXIDE MT-100 TV | Titanium Dioxide & Aluminum Hydroxide & Stearic Acid | 5 | 5 |
| PRIMROSE-TRI-77891 | TITANIUM DIOXIDE & SILICA | 6.5 | 6.5 |
| PRIMROSE-Y-77492 | IRON OXIDES & SILICA | 0.8 | 0.8 |
| PRIMROSE-R-77491 | IRON OXIDES & SILICA | 0.3 | 0.3 |
| PRIMROSE-B-77499 | IRON OXIDES & SILICA | 0.1 | 0.1 |
| ZEMEA | PROPANEDIOL | 10 | 10 |
| PHENOXETOL NF 272556 | PHENOXYETHANOL | 0.5 | 0.5 |
| RHEOZAN | SUCCINOGLYCAN | 0.5 | 0.5 |
| Property | Ratio of Neutralization agent/fatty acids | 0.10 | 0.13 |
| | | Uniform | Uniform |
| | Appearance | cream | cream |
| | Hardness (mN) | 65 | 15 |
| | In vitro UV protective effect | + | +/− |
| | Matteness | + | + |
| | Spreadability | + | + |

Results of Table 7: Incorporating inorganic UV filters and pigments made SPF complexion with sufficient UV protective effect and Matte finish.

TABLE 8

| | | Example | Example | Example |
|---|---|---|---|---|
| Material name En | INCI Name | 14 | 15 | 16 |
| DEIONIZED WATER | WATER | 40.5 | 40.5 | 35.5 |
| GLYCERINE NAT 99.7% ACIDCHEM | GLYCERIN | 5 | 5 | 5 |
| CAUSTIC POTASH DRY | POTASSIUM HYDROXIDE & WATER | 0.5 | 0.5 | 0.5 |
| STEARIC ACID POFAC | STEARIC ACID | 8 | 8 | 8 |
| NAA-222S BEADS (JSQI) | BEHENIC ACID | 2 | 2 | 2 |
| LANETTE ® 22 | BEHENYL ALCOHOL | 1 | 1 | 1 |
| NEO HELIOPAN OS OCTISLTE | ETHYLHEXYL SALICYLATE | 5 | 5 | 5 |
| PARSOL ® 340 | OCTOCRYLENE | | 5 | 5 |
| NEO HELIOPAN HMS HOMSLTE | HOMOSALATE | 10 | | |
| BOTANESSENTIAL OLIVE S-EHO | ETHYLHEXYL OLIVATE, SQUALANE | 5 | 10 | 10 |
| TITANIUM DIOXIDE MT-100 TV | Titanium Dioxide & Aluminum | | | 5 |
| | Hydroxide & Stearic Acid | | | |
| PRIMROSE-TRI-77891 | TITANIUM DIOXIDE & SILICA | 10.000 | 10.000 | 10.000 |
| PRIMROSE-Y-77492 | IRON OXIDES & SILICA | 9.000 | 9.000 | 9.000 |
| PRIMROSE-R-77491 | IRON OXIDES & SILICA | 3.000 | 3.000 | 3.000 |
| PRIMROSE-B-77499 | IRON OXIDES & SILICA | 0.500 | 0.500 | 0.500 |
| ZEMEA | PROPANEDIOL | 10 | 10 | 10 |
| PHENOXETOL NF 272556 | PHENOXYETHANOL | 0.5 | 0.5 | 0.5 |
| RHEOZAN | SUCCINOGLYCAN | 0.5 | 0.5 | 0.5 |
| Property | Ratio of Neutralization agent/fatty acids | 0.05 | 0.05 | 0.05 |
| | Appearance | Uniform | Uniform | Uniform |
| | | cream | cream | cream |
| | Hardness (mN) | 102 | — | 117 |
| | Viscosity (mPa · s) | — | 30,000 | — |
| | In vitro UV protective effect | +/− | +/− | + |
| | Matteness | + | + | + |
| | Spreadability | + | + | + |

Results of Table 8: This complexion base worked well with different UV filters.

TABLE 9

| | | Example | Example | Example |
|---|---|---|---|---|
| Material name En | INCI Name | 17 | 18 | 19 |
| DEIONIZED WATER | WATER | 28.5 | 26 | 47.4 |
| GLYCERINE NAT 99.7% ACIDCHEM | GLYCERIN | 5 | 5 | 5 |
| CAUSTIC POTASH DRY | POTASSIUM HYDROXIDE & WATER | 0.5 | 0.5 | 0.5 |
| STEARIC ACID POFAC | STEARIC ACID | 4 | 4 | 3 |
| NAA-222S BEADS (JSQI) | BEHENIC ACID | 1 | 1 | 0.5 |
| LANETTE ® 22 | BEHENYL ALCOHOL | 0.5 | 0.5 | 0.4 |
| NEO HELIOPAN OS OCTISLTE | ETHYLHEXYL SALICYLATE | | | 5 |
| PARSOL ® 340 | OCTOCRYLENE | | | 5 |
| BOTANESSENTIAL OLIVE S-EHO | ETHYLHEXYL OLIVATE, SQUALANE | 15 | 15 | 5 |
| VEGELIGHT C912-LC | C9-12 ALKANE, COCO-CAPRYLATE/CAPRATE | 15 | 15 | |
| TITANIUM DIOXIDE MT-100 TV | Titanium Dioxide & Aluminum Hydroxide & Stearic Acid | 7.5 | 10 | 5 |
| PRIMROSE-TRI-77891 | TITANIUM DIOXIDE & SILICA | 6.5 | 6.5 | 6.5 |
| PRIMROSE-Y-77492 | IRON OXIDES & SILICA | 0.8 | 0.8 | 0.8 |
| PRIMROSE-R-77491 | IRON OXIDES & SILICA | 0.3 | 0.3 | 0.3 |
| PRIMROSE-B-77499 | IRON OXIDES & SILICA | 0.1 | 0.1 | 0.1 |
| ZEMEA | PROPANEDIOL | 10 | 10 | 10 |
| PHENOXETOL NF 272556 | PHENOXYETHANOL | 0.5 | 0.5 | 0.5 |
| RHEOZAN | SUCCINOGLYCAN | 0.5 | 0.5 | 0.5 |
| Property | Ratio of Neutralization agent/fatty acids | 0.10 | 0.10 | 0.14 |
| | Appearance | Uniform | Uniform | Uniform |
| | | cream | cream | cream |
| | Hardness (mN) | 15 | 27 | 15 |
| | In vitro UV protective effect | +/− | + | + |
| | Matteness | + | + | + |
| | Spreadability | + | + | + |

Results of Table 9: This complexion base worked well with different UV filters and inorganic SPF system.

### Example E

A number compositions were tested to determine the effect a content of fatty alcohol and/or water soluble amphiphilic agent(s) on the composition. It was determined that less than 1 mass % of fatty alcohol and/or water soluble amphiphilic agent(s) results in compositions with less soapy white case. The tested compositions are summarized in Tables 10-11. The tested compositions were prepared using a manufacturing process which was the same or similar to the manufacturing processes of Examples B-D. The tested compositions were evaluated for a number of properties using evaluation procedures, which are the same or similar to the evaluation procedures of Examples B-D.

TABLE 10

| | | Exam- | Exam- | Exam- | Exam- | Exam- | Exam- | Exam- |
|---|---|---|---|---|---|---|---|---|
| Material name En | INCI Name | ple 20 | ple 21 | ple 22 | ple 23 | ple 24 | ple 25 | ple 26 |
| DEIONIZED WATER | WATER | 59 | 59 | 58.5 | 59 | 56 | 66.75 | 69.05 |
| GLYCERINE NAT 99.7% | GLYCERIN | 5 | 5 | 5 | 5 | 5 | 5 | 5 |
| ACIDCHEM | | | | | | | | |
| CAUSTIC POTASH DRY | POTASSIUM HYDROXIDE | 0.4 | 0.4 | 0.5 | 0.5 | 0.5 | 0.25 | 0.15 |
| | & WATER | | | | | | | |
| STEARIC ACID POFAC | STEARIC ACID | 8 | 8 | 8 | 8 | 8 | 2.5 | 1.5 |
| NAA-222S BEADS (JSQI) | BEHENIC ACID | 2 | 2 | 2 | 2 | 2 | 0.5 | 0.5 |
| LANETTER ® 22 | BEHENYL ALCOHOL | 2 | 1 | 0.5 | | 1 | 0.5 | 0.3 |
| BOTANESSENTIAL OLIVE | ETHYLHEXYL OLIVATE, | 10 | 10 | 10 | 10 | 10 | 5 | 5 |
| S-EHO | SQUALANE | | | | | | | |
| Lexgard ™ Natural MHG MB | Methylheptylglycerin | | | | | 1 | 1 | 1 |
| ZEMEA | PROPANEDIOL | 10 | 10 | 10 | 10 | 10 | 10 | 10 |
| PHENOXETOL NF 272556 | PHENOXYETHANOL | 0.5 | 0.5 | 0.5 | 0.5 | 0.5 | 0.5 | 0.5 |
| RHEOZAN | SUCCINOGLYCAN | 0.5 | 0.5 | 0.5 | 0.5 | 0.5 | 0.5 | 0.5 |
| Property | Ratio of Neutralization | 0.04 | 0.04 | 0.05 | 0.05 | 0.05 | 0.08 | 0.08 |
| | agent/fatty acids | | | | | | | |
| | Appearance | Uniform | Uniform | Uniform | Uniform | Uniform | Uniform | Uniform |
| | | cream | cream | cream | cream | cream | cream | cream |
| | Hradness (mN) | 35 | 40 | 79 | 212 | 24 | 10 | |
| | Viscosity (mPa · s) | | | | | | | 11,300 |
| | Blurring effect | + | + | + | + | + | +/− | +/− |
| | Spreadability | + | + | + | + | + | + | + |
| | Soapy white cast during | − | +/− | + | + | + | + | + |
| | application | | | | | | | |

Results of Table 10: Less than 1% fatty alcohol and Methylhepthylglycerin improved soapy white cast effectively in skincare formulas.

TABLE 11

| | | Example | Example | Example |
|---|---|---|---|---|
| Material name En | INCI Name | 27 | 28 | 29 |
| DEIONIZED WATER | WATER | 41.4 | 41.4 | 41.4 |
| GLYCERINE NAT 99.7% ACIDCHEM | GLYCERIN | 5 | 5 | 5 |
| STEARIC ACID POFAC | STEARIC ACID | 4 | 4 | 3 |
| NAA-222S BEADS (JSQI) | BEHENIC ACID | 1 | 1 | 0.5 |
| LANETTE ® 22 | BEHENYL ALCOHOL | 0.5 | 0.5 | 0.4 |
| CAUSTIC POTASH DRY | POTASSIUM HYDROXIDE & WATER | 0.5 | 0.5 | 0.5 |
| NEO HELIOPAN OS OCTISLTE | ETHYLHEXYL SALICYLATE | 5 | 5 | 5 |
| PARSOL ® 340 | OCTOCRYLENE | 5 | 5 | 5 |
| BOTANESSENTIAL OLIVE S-EHO | ETHYLHEXYL OLIVATE, SQUALANE | 5 | 5 | 5 |
| TITANIUM DIOXIDE MT-100 TV | Titanium Dioxide & Aluminum Hydroxide & Stearic Acid | 5 | 5 | 5 |
| PRIMROSE-TRI-77891 | TITANIUM DIOXIDE & SILICA | 7.0 | 7.0 | 7.0 |
| PRIMROSE-Y-77492 | IRON OXIDES & SILICA | 1.0 | 1.0 | 1.0 |
| PRIMROSE-R-77491 | IRON OXIDES & SILICA | 0.3 | 0.3 | 0.3 |
| PRIMROSE-B-77499 | IRON OXIDES & SILICA | 0.1 | 0.1 | 0.1 |
| ZEMEA | PROPANEDIOL | 10 | 10 | 10 |
| PHENOXETOL NF 272556 | PHENOXYETHANOL | 0.5 | 0.5 | 0.5 |
| Lexgard Natural MHG MB | Methylheptylglycerin | | 1 | 1 |
| RHEOZAN | SUCCINOGLYCAN | 0.5 | 0.5 | 0.5 |
| DEIONIZED WATER | WATER | 5 | 5 | 5 |
| Property | Ratio of Neutralization agent/fatty acids | 0.10 | 0.10 | 0.14 |
| | Appearance | Uniform | Uniform | Uniform |
| | | cream | cream | cream |
| | Viscosity (mPa · s) | 73,000 | 49450 | 27350 |
| | In vitro UV protective effect | + | + | + |
| | Matteness | + | + | + |
| | Spreadability | + | + | + |
| | Soapy white cast during application | +/− | + | + |

Results of Table 11: Less than 1% fatty alcohol and Methylhepthylglycerin improved soapy white cast effectively in complexion formulas.

### Example F

Additional compositions presented in Tables 12 and 13 were prepared using using a manufacturing process which was the same or similar to the manufacturing processes of Examples B-D.

TABLE 12
SPF cream foundation

| | | Example | Example | Example |
|---|---|---|---|---|
| Material name En | INCI Name | 27 | 28 | 29 |
| DEIONIZED WATER | WATER | To 100 | To 100 | To 100 |
| GLYCERINE NAT 99.7% ACIDCHEM | GLYCERIN | 10 | 10 | 10 |
| PALMITIC ACID S 02053 | Palmitic Acid | 5 | 3 | 5 |
| MYRISTIC ACID | Myristic Acid | 3 | 5 | 5 |
| BATYL ALCOHOL EX | Batyl Alcohol | 0.9 | 0.9 | 0.9 |
| CAUSTIC POTASH DRY | POTASSIUM HYDROXIDE & WATER | 0.6 | 0.6 | 0.6 |
| NEO HELIOPAN OS | ETHYLHEXYL SALICYLATE | 5 | 5 | 5 |
| OCTISLTE | | | | |
| PARSOL ® 340 | OCTOCRYLENE | 10 | 10 | 10 |
| BOTANESSENTIAL OLIVE S-EHO | ETHYLHEXYL OLIVATE, SQUALANE | 5 | 5 | 5 |
| TAYCA MP-25 MG | TITANIUM DIOXIDE & MAGNESIUM | 2.0 | 2.0 | 2.0 |
| | STEARATE | | | |
| UNIPURE YELLOW LC | IRON OXIDES (duplicate 2) & | 1.0 | 1.0 | 1.0 |
| 182 LL | LAUROYL LYSINE | | | |
| UNIPURE RED LC 381 | IRON OXIDES (duplicate 1) & | 0.3 | 0.3 | 0.3 |
| LL | LAUROYL LYSINE | | | |
| UNIPURE BLACK LC | IRON OXIDES (duplicate 3) & | 0.1 | 0.1 | 0.1 |
| 989 LL | LAUROYL LYSINE | | | |
| ZEMEA | PROPANEDIOL | 10 | 10 | 10 |
| PHENOXETOL NF 272556 | PHENOXYETHANOL | 0.5 | 0.5 | 0.5 |
| LEXGARD E | Ethylhexylglycerin | 2 | 2 | 2 |
| KELTROL (RM-09121) | XANTHAN GUM | 0.5 | 0.5 | 0.5 |

TABLE 13
A skincare product

| | | Example | Example | Example |
|---|---|---|---|---|
| Material name En | INCI Name | 30 | 31 | 32 |
| DEIONIZED WATER | WATER | To 100 | To 100 | To 101 |
| GLYCERINE NAT 99.7% | GLYCERIN | 8 | 8 | 8 |
| ACIDCHEM | | | | |
| ZEMEA | PROPANEDIOL | 5 | 10 | 10 |
| PHENOXETOL NF 272556 | PHENOXYETHANOL | 0.5 | 0.5 | 0.5 |
| SANGELOSE 90L | HYDROXYPROPYL | 0.5 | 0.3 | 0.3 |
| | METHYLCELLULOSE STEAROXY | | | |
| | ETHER | | | |
| AQUALON ™ CMC 7M2F | CELLULOSE GUM | 0.3 | 0.3 | 0.3 |
| CAUSTIC POTASH DRY | POTASSIUM HYDROXIDE & WATER | 0.8 | 0.5 | 0.5 |
| STEARIC ACID POFAC | STEARIC ACID | 15 | 8 | 5 |
| MYRISTIC ACID | Myristic Acid | 5 | 5 | 2 |
| BATYL ALCOHOL EX | Batyl Alcohol | 1 | 0.3 | 0.5 |
| ESERAN 200 | DIISOPROPYL SEBACATE | 10 | 10 | 10 |
| NEO HELIOPAN OS | ETHYLHEXYL SALICYLATE | 2 | 2 | 2 |
| OCTISLTE | | | | |

Although the foregoing refers to particular preferred embodiments, it will be understood that the present invention is not so limited. It will occur to those of ordinary skill in the art that various modifications may be made to the disclosed embodiments and that such modifications are intended to be within the scope of the present invention.

All of the publications, patent applications and patents cited in this specification are incorporated herein by reference in their entirety.

1. An oil-in-water type emulsified cosmetic composition comprising:
(a) at least one fatty acid; and
(b) at least one neutralization agent, wherein a mass ratio between the at least one neutralization agent and the at least one fatty acid is from 0.001 to 0.2.

2. An oil-in-water type emulsified cosmetic composition comprising:
(a) a thickener comprising at least one natural polymer; and
(b) at least one fatty acid;
(c) at least one neutralization agent, which partially neutralizes carboxylic acid groups of the at least one fatty acid, wherein the composition does not contain a polyethylene glycol based ingredient.

3. The composition of claim 1, wherein the at least one fatty acid comprises at least one of stearic acid and behenic acid.

4. The composition of claim 1, wherein an amount of the at least fatty acid is from 0.1 mass % to 40 mass % of the composition.

5. The composition of claim 1, wherein the at least one neutralizing agent comprises at least one water soluble base.

6. The composition of claim 5, wherein the at least one water soluble base comprises at least one of potassium hydroxide and sodium hydroxide.

7. The composition of claim 1, wherein an amount of the at least one neutralizing agent is from 0.01 mass % to 5 mass % of the composition.

8. The composition of claim 1, further comprising at least one natural polymer.

9. The composition of claim 2, wherein the at least one natural polymer is selected from succinoglycan, sclerotium gum, xanthan gum, a cellulose derivative or a combination thereof.

10. The composition of claim 2, wherein an amount of the at least one natural polymer is from 0.01 mass % to 5 mass % of the composition.

11. The composition of claim 1 further comprising UV scattering particles.

12. The composition of claim 11, wherein the UV scattering particles comprise titanium oxide particles, zinc oxide particles or a combination thereof.

13. The composition of claim 11, wherein an amount of the UV scattering particles is from 0.5 mass % to 30 mass % of the composition.

14. The composition of claim 1 further comprising one or more of organic UV absorbers.

15. The composition of claim 1, wherein an amount of the one or more of organic UV absorbers is from 0.5 mass % to 30 mass %.

16. The composition of claim 1, further comprising one or more pigments.

17. The composition of claim 16 wherein the one or more pigments comprises water dispersible pigments.

18. The composition of claim 16 wherein the one or more pigments comprise hydrophobically treated pigments.

19. The composition of claim 16, wherein the one or more pigments comprise iron oxide.

20. The composition of claim 16, wherein an amount of the one or more pigments is from 0.05 mass % to 25 mass % of the composition.

21. The composition of claim 1, further comprising at least one non-PEG water soluble amphiphilic agent.

22. The composition of claim 21, wherein the at least one non-PEG water soluble amphiphilic agent comprises a fatty alcohol.

23. The composition of claim 21, wherein an amount of the at least one non-PEG soluble amphiphilic agent is from 0.01 mass % to less 5 mass % of the composition.

24. The composition of claim 1, wherein the composition does not contain a polyethylene glycol based ingredient.

25. The composition of claim 1, wherein the composition does not contain microplastic particles.

26. The composition of claim 1, wherein the composition further comprises at least one preservative agent.

27. The composition of claim 26, wherein the at least one preservative agent comprises at least one amphiphilic liquid preservative agent.

28. The composition of claim 27, wherein the at least one amphiphilic liquid preservative agent comprises methylheptylglycerin, ethylheptylglycerin or a combination thereof.

29. The composition of claim 27, wherein an amount of the at least one amphiphilic liquid preservative agent is from 0.01 mass % to 5 mass % of the composition.

30. The composition of claim 27, wherein the at least one preservative agent further comprises phenoxyethanol.

31. The composition of claim 30, wherein an amount of phenoxyethanol is from 0.01 mass % to 1.5 mass % of the composition

32. The composition of claim 1 comprising:
0.1 mass % to 40 mass % of at least one of stearic acid and behenic acid;
0.01 mass % to 5 mass % of at least one of potassium hydroxide or sodium hydroxide;
0.01 mass % to 5 mass of fatty alcohol;
0.01 mass % to 5 mass % of succinoglycan;
0.01 mass % to 5 mass % of methylheptylglycerin, ethylheptylglycerin or a combination thereof;
0.01 mass % to 1.5 mass % of phenoxyethanol; and
at least one of (i) 0.5 mass % to 30 mass % of titanium oxide particles, zinc oxide particles or a combination thereof; (ii) 0.5 mass % to 30 mass % of one or more organic UV absorbers and (iii) 0.05 mass % to 25 mass % of pigments.

33. The composition of claim 1 having at least one a hardness greater than 4 mN and a viscosity of at least 10,000 mPa·s.

34. A cosmetic method comprising applying the cosmetic composition of claim 1 to a keratinous surface.
